(19) United States
(12) Patent Application Publication
(10) Pub. No.: US 2009/0062750 A1
(43) Pub. Date: Mar. 5, 2009

(54) MEDICAL DEVICE FOR INJECTING A LOCAL ANESTHETIC

(76) Inventor: Morteza Gharib, Hollidaysburg, PA (US)

Correspondence Address: MORTEZA GHARIB, 204 WOODLAWN TERRACE, HOLLIDAYSBURG, PA, 16648, US

(21) Appl. No.: 12/189,661

(22) Filed: Aug. 11, 2008

Related U.S. Application Data
(60) Provisional application No. 60/968,166, filed on Aug. 27, 2007.

(51) Int. Cl. A61M 5/32; A61M 5/315

(52) U.S. Cl. 604/218; 604/180

(57) ABSTRACT

An improved medical device for injecting a local anesthetic into an organism. The device comprises a needle, an injection tube attached to the posterior end of the needle, a plunger attached to the posterior end of the needle, a housing dimensioned so that the plunger slides through the housing with friction, and a conduit dimensioned to allow the needle to pass through. The device allows an anesthesia provider to perform a nerve block more safely and efficiently. In one embodiment of the device, the nerve block can be performed without an assistant.

### CROSS-REFERENCE

This application claims the benefit, under 35 U.S.C. §119(e)(1), of U.S. provisional patent application No. 60/968,166 filed on Aug. 27, 2007, titled “A Regional Anesthesia Needle Assembly for an Easy and Safe Performance of a Nerve Block.”

### FIELD OF THE INVENTION

The invention relates generally to medical devices for injecting fluid solutions into an organism and more particularly to medical devices for injecting a local anesthetic.

### BACKGROUND

A nerve block is a procedure in which an anesthesia provider will place a special insulated needle in very close proximity to a nerve within the human body and perhaps animals. A local anesthetic solution is injected through the needle and around the nerve in order to provide anesthesia and pain relief in an area of the body which is innervated by the targeted nerve.

In order to accomplish this procedure correctly, a majority of anesthesia providers use traditional nerve block needles which consist of an insulated steel shaft needle. At the rear end the needle is attached to an injection tube and a conductive wire.

A small battery-operated nerve stimulator device is attached to the rear end of the conductive wire. The anesthesia provider will advance the needle through the skin toward the targeted nerve. An assistant operates the nerve stimulator by sending decreasing amounts of electric current through the needle, causing a specific muscle contraction. This contraction indicates the proximity of the needle to that specific nerve. Then the assistant injects the local anesthetic solution into the injection port. The solution goes through the tubing and the needle to be deposited near the desired nerve.

During the whole procedure the anesthesia provider needs both hands to keep the needle in a constant direction and depth, and also to advance the needle very gently and smoothly. This process is necessary in order to prevent nerve injury or accidental injection of the medicine inside a blood vessel or a nerve. Either one of which can have grave consequences.

It is known in the art that performance of a nerve block: (1) is a very delicate procedure, (2) can be time consuming, (3) takes a great amount of practice to master, (4) almost always requires the presence of an assistant, (5) can have a significant failure rate, (6) can result in the fatigue of the anesthesia provider's hands, arms, and back, (7) can result in accidental nerve injury or intravascular injection which can lead to grave consequences such as seizure, coma, and even death, (8) the needle can become dislodged as a result of the slightest hand movements, and (9) has the potential to cause needle injury to the anesthesia provider and medical assistants.

Today, more and more people are using ultrasound imaging devices to help them visualize the nerve during the performance of a nerve block. This requires use of bulky and expensive devices which will not eliminate the need for an assistant.

There is a need in the field for a medical device for injecting a local anesthetic that reduces the risk of the negative outcomes mentioned above, is easier to use than similar devices in the field today, and has the potential of allowing an anesthesia provider to effectively locate a nerve and inject the local anesthetic without the use of an assistant.

### SUMMARY

The present invention is directed to a device that satisfies these needs of reduction in the risk of negative outcomes associated with nerve block procedures, making the nerve block procedure easier to perform, and creating the potential for allowing an anesthesia provider to effectively inject the local anesthetic without the use of an assistant. The device generally comprises a conventional regional anesthesia needle, a plunger, an injection tube, an injection port, a housing and a conduit. The injection port and the needle being attached to opposite ends of the injection tube. The housing is shaped with dimensions that allow the plunger to slide inside the housing with a certain amount of friction created between the plunger and the housing. The conduit is positioned at the front opening of the housing. The conduit guides the needle as the needle moves from a position where the needle's length is inside the housing to a position where needle is projecting out from the front of the housing.

In an alternative embodiment, present invention further comprises an injection pad. The base of injection pad is coated with a sticky material that allows the injection pad to be temporarily attached to the patient's skin. The injection port at the end of the injection tube is attached to the top surface of the injection pad to provide stable injection port so that the anesthetic solution can be single-handedly directed into the injection tube.

In order to place a nerve block, the anesthesia provider holds the conduit between the thumb and index finger. The conduit front end is rested against the patient's skin over the targeted nerve. The conductive wire is attached to the nerve stimulator. The anesthesia provider uses one hand to constantly hold the conduit against the patient's skin. The other hand is used alternatively to advance the plunger and also to operate the nerve stimulator. By advancing the plunger, the needle inside the housing moves toward the targeted nerve. After the targeted nerve is located, the anesthesia provider has the option of using an assistant to inject the local anesthetic solution into the injection port, or as it was described in the above alternate embodiment, the provider can use the specifically designed injection pad to single handedly inject the local anesthetic into injection port.

At the end of the procedure, the anesthesia provider will retract the plunger and hence the needle inside the housing, and disposes of the needle safely. This will reduce the risk of needle injury to the anesthesia provider and other medical personnel.

These and any other features, aspects, and advantages of the present invention will become better understood with regard to the following description, appended claims, and accompanying drawings.

### DRAWINGS

FIG. 1 shows a prior art nerve block needle.

FIG. 2 shows a sectional side view of a version of the plunger for the invention.

FIG. 3 shows a sectional side view of a version of the housing for the invention.

FIG. 4 shows a sectional side view of a version of the invention.

FIG. 5 shows a sectional side view of a version of the invention.

FIG. 5a shows a sectional side view of a version of the conduit for the invention.

FIG. 6 shows a sectional side view of a version of the plunger for the invention.

FIG. 7 shows an exploded sectional side view of a version of the plunger for the invention.

FIG. 8 shows a plan horizontal view of a version of the injection pad for the invention.

FIG. 9 shows a sectional side view of a version of the injection pad for the invention.

### DESCRIPTION

Referring to FIG. 1, a conventional nerve block device is comprised of a hollow needle 30 that is connected to an injection tube 34 and a conductive wire 36. The injection tube 34 has an injection port 37 at its posterior end. The injection port 37 is used to introduce the local anesthetic into the injection tube 34. The posterior end of the conductive wire 36 may be connected to a battery operated nerve stimulator device. The conductive wire 36 enables pulses of electricity to be sent through the needle 30 in order to determine the distance between the needle 30 and the nerve that the anesthesia provider desires to block.

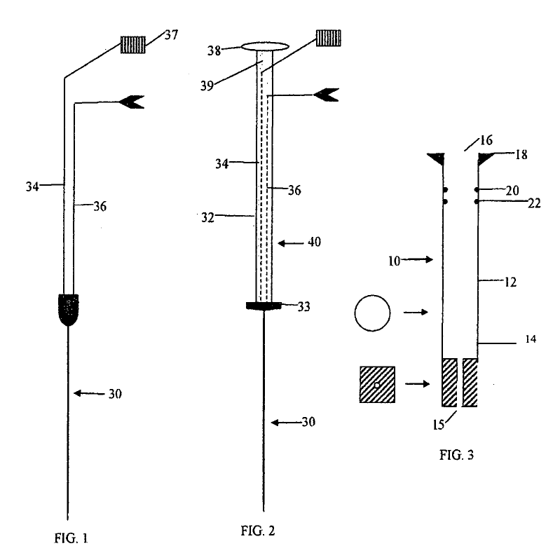

In the preferred embodiment of the plunger 40 shown in FIG. 2, the plunger 40 comprises a shaft 32, a posterior knob 38, and an anterior ring 33. The needle 30 is mounted to the front side of the anterior ring 33. Conductive wire 36 and the injection tube 34 are threaded through the shaft 32 and exit the posterior end of the shaft 32. The plunger 40 can be constructed out of any solid material, but is preferably made of plastic.

In the embodiment of the housing 10 shown in FIG. 3, the housing 10 comprises a cylindrical body 12, a anterior end 14, and posterior end 16. The housing 10 has a finger flange 18 at its posterior end 16. The conduit 15 is attached to the anterior end 14 of the housing 10. A rear stop flange 20 and a front stop flange 22 are positioned inside the housing 10. The rear and front stop flange 20, 22 maintain the plunger 40 inside the housing 10. The housing 10 can be any shape, but is preferably cylindrical. In addition, the housing 10 can be made of any solid material, but is preferably made of clear plastic to allow the user to see the needle 30 inside the housing 10.

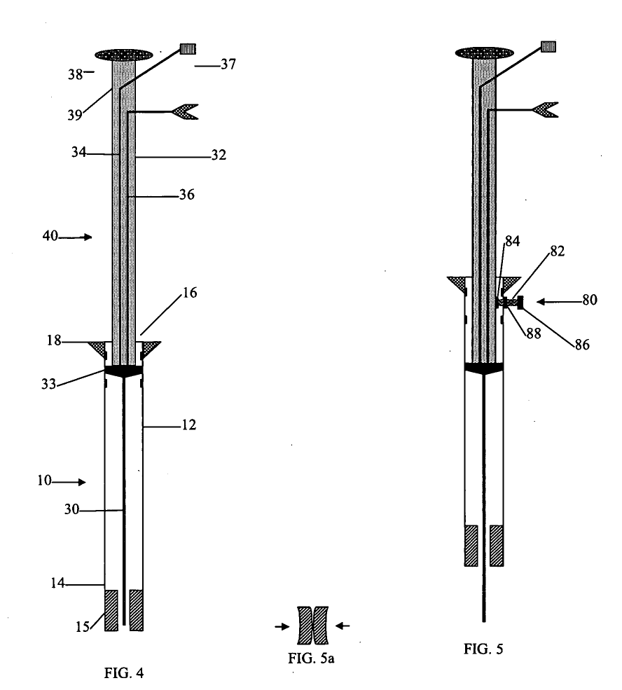

Referring to the embodiment of the invention shown in FIG. 4, the plunger 40 is inserted into the chamber 10 in such a way as to allow the needle 30 to pass through the conduit 15. The internal diameter of the conduit 15 is made slightly larger than the outer diameter of the needle 30. This allows the needle 30 to pass through the conduit 15 in a guided fashion. The anterior ring 33 of the plunger 40 is placed between the front and rear stop flange 20 and 22. The injection tube 34 and the conductive wire 36 are threaded through the shaft 32 of the plunger 40. The posterior knob 38 of the plunger 40 is used by the anesthesia provider to force the needle 30 through the conduit 15 and into the organism that will receive the local anesthetic.

Referring to the embodiment of the invention shown in FIG. 5, a locking mechanism 80 may be attached to the housing to lock the needle in a position. The embodiment of the locking mechanism shown in FIG. 5 comprises a shaft 82, a knob 86, and a break pad 84. The shaft 82 and knob 86 can be made of any solid material like plastic or metal. The shaft 82 is threaded on the surface and functions like a standard bolt. The break pad 84 can be made of any elastic material such as rubber. As shown in FIG. 5, the embodiment of the locking mechanism 80 comprises a threaded nut 88. The threaded nut 88 functions like a standard hardware nut thereby allowing the shaft 82 to be screwed in and out of the threaded nut 88, thereby moving the break pad 84 up to and away from the plunger inside the housing.

Referring to the embodiment of the invention shown in FIG. 5a, the locking mechanism can comprise a conduit at the front of the housing that is made of a soft, elastic material such as plastic or rubber. When a user applies pressure to the conduit, the conduit narrows and creates friction against the needle. Applying pressure in this way allows the user to hold the needle in a desired position. By releasing the pressure, the user can then move the needle again.

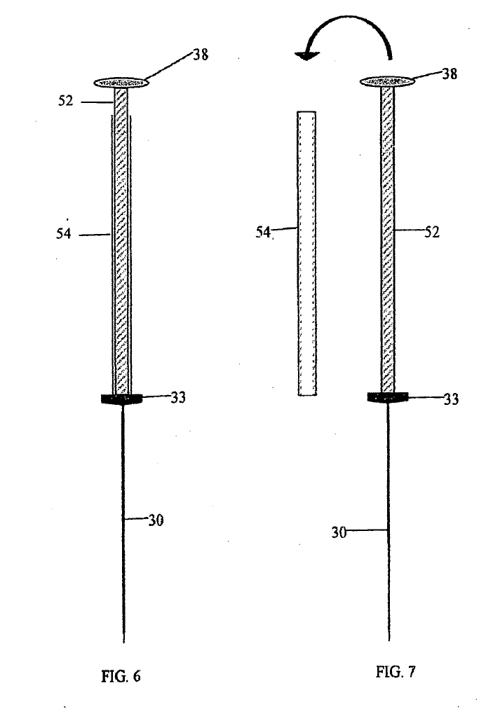

In an alternative embodiment of the invention shown in FIGS. 6 and 7, the invention comprises a fine tuning mechanism. As illustrated in FIGS. 6 and 7, the embodiment of the plunger 40 comprises two hollow cylinders 52, 54. The inner cylinder 52 is threaded on its outer surface, and the outer cylinder 54 is threaded on its inner surface. They function like a standard bolt and nut. The two cylinders 52, 54 move telescopically against each other by twisting both cylinders 52, 54 in opposite directions. In practice, the outer cylinder 54 is locked into place against the housing using locking mechanism 80. A user then rotates the inner cylinder 52 using the knob 38 that is attached to the inner cylinder 52. The rotation of the inner cylinder 52 causes the inner cylinder 52 to advance forward, away from the outer cylinder 54. Since the needle 30 is attached to the inner cylinder 52, the needle 30 advances in a forward direction. The fine tuning mechanism allows a user to achieve very fine movements of the needle 30 when the needle 30 is inserted into the organism.

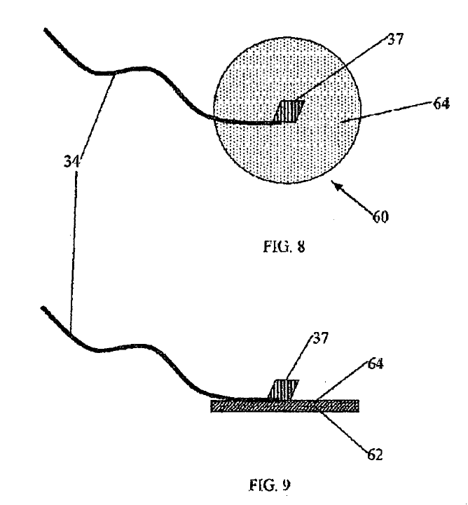

An alternative embodiment of the invention comprises an injection pad. Embodiments of the injection pad are illustrated in FIGS. 8 and 9. Referring to FIG. 8, the injection pad 60 can be made of any flexible material such as plastic or rubber. The injection pad 60 has a top surface 64 and a bottom surface 62. An injection port 37 is attached to the top surface 64 of the injection pad 60. The bottom surface 62 is covered with a layer of adhesive substance, which is preferably covered with a removable backing. When a user removes the backing, the injection pad 60 can be attached to a patient's body or any available surface near the patient. The injection pad 60 provides a stable, immobile injection port 37, which allows the anesthesia provider to single-handedly attach, inject, and detach syringes of local anesthetics into the injection port 37. The injection pad 60 can be made in any size and shape, but is preferably made in the shape of a disc or square.

What is claimed is:

1. A medical device for injecting a local anesthetic into an organism, said device comprising:
(a) a needle having an anterior end and a posterior end;
(b) an injection tube attached to said posterior end of said needle;
(c) a plunger attached to said posterior end of said needle;
(d) a housing having an anterior end and a posterior end, said housing being dimensioned to allow said plunger to move inside said housing with friction; and
(e) a conduit positioned at said anterior end of said housing, said conduit being dimensioned to allow said needle to move through said conduit.

2. The medical device of claim 1, wherein said needle is an insulated needle.

3. The medical device of claim 1, wherein said needle is an un-insulated needle.

4. The medical device of claim 2, wherein said device further comprises a conductive wire attached to said posterior end of said needle.

5. The medical device of claim 1, further comprising a locking means for locking said needle in position.

6. The medical device of claim 5, wherein said locking means comprises a screw and nut.

7. The medical device of claim 5, wherein said locking means comprises elastic conduit.

8. The medical device of claim 1, further comprising an injection pad, said injection pad comprised of a top surface, a bottom surface, and an injection port.

9. The medical device of claim 8, wherein said injection port is attached to said top surface.

10. The medical device of claim 8, wherein said bottom surface is covered with layer of adhesive material.

11. A method of using a medical device for injecting a local anesthetic into an organism, the device having:
(a) a needle having an anterior end and a posterior end;
(b) an injection tube attached to said posterior end of said needle, said injection tube having an injection port at its anterior end;
(c) a conductive wire attached to said posterior end of said needle;
(d) a plunger attached to said posterior end of said needle;
(e) a housing having an anterior end and a posterior end, said housing being dimensioned to allow said plunger to move inside said housing with friction;
(f) a conduit positioned at said anterior end of said housing, said conduit being dimensioned to allow said needle to move through said conduit;
(g) an injection pad having sticky bottom surface and accepting said injection port on its top surface;
the method comprising:
attaching said conductive wire to a nerve stimulator device;
attaching said injection pad to an available surface or to a patient body;
holding said conduit with one hand against patient's body;
advancing said plunger and so said needle using the other hand;
using said other hand alternatively to advance said plunger and also operate said nerve stimulator;
using said injection pad to attach syringe of local anesthetic to injection port;
injecting local anesthetics into the injection port.

12. The method of claim 11 wherein advancing plunger and so the needle, operating the nerve stimulator and injecting the syringes of local anesthetics into injection port are performed using one hand while the other hand is constantly holding conduit against the patient's skin.

13. The method of claim 11 wherein the plunger and so the needle are pulled back into the housing after placement of the nerve block.

14. The method of claim 11 wherein placement of a nerve block is performed without an assistant.
